(19) United States
(12) Patent Application Publication
(10) Pub. No.: US 2013/0144156 A1
(43) Pub. Date: Jun. 6, 2013

(54) Method and Apparatus for Correcting B1-Inhomogeneity in Slice-Selective Nuclear Magnetic Resonance Imaging

(71) Applicant: Nicolas Boulant, Saint Aubin (FR)

(72) Inventor: Nicolas Boulant, Saint Aubin (FR)

(73) Assignee: Commissariat A L'Energie Atomique Et Aux Energies Alternatives, Paris (FR)

(21) Appl. No.: 13/698,480

(22) PCT Filed: May 21, 2010
(86) PCT No.: PCT/IB10/01479

§ 371 Date: Feb. 22, 2013

(51) Int. Cl. A61B 5/055

(52) U.S. Cl. CPC A61B 5/055
USPC 600/410

(57) ABSTRACT

A method of performing nuclear magnetic resonance imaging of a body, comprising: immerging said body in a static magnetic field for aligning nuclear spins along a magnetization axis; exposing said body to a gradient pulse and to a transverse radio-frequency pulse for performing slice-selective excitation of said nuclear spins, thus flipping the nuclear spins of atoms contained within a slice of said body; detecting a signal emitted by excited nuclear spins; and reconstructing a magnetic resonance image of said slice of the body on the basis of the detected signal; the method being characterized in that said radio-frequency pulse is constituted by a train of slice-selective elementary pulses, approximately equivalent to a train of elementary rectangular pulses with constant frequencies which are designed for compensating for inhomogeneity of the radio-frequency field within the body.

The invention relates to a method for correcting the radio-frequency (or “B₁”) spatial inhomogeneity in slice-selective nuclear magnetic resonance imaging. The invention also relates to an apparatus, or “scanner” for carrying out such a method. The invention applies notably, but not exclusively, to the field of medical imaging.

Magnetic resonance imaging (MRI) is a very powerful tool in research and diagnostics. It comprises immerging a body in a static magnetic field B₀ for aligning nuclear spins thereof; exposing it to a transverse radio-frequency (RF) field B₁ (excitation sequence) at a resonance frequency known as the “Larmor frequency” for flipping said nuclear spins by a predetermined angle; and detecting a signal emitted by flipped nuclear spins, from which an image of the body can be reconstructed.

There is a trend to move towards higher and higher static magnetic fields in order to improve the spatial resolution of MRI. For example, magnetic fields of 1.5 T (Tesla) are currently used in clinical practice, 3 T is the highest field used in commercial apparatuses and research systems can operate at more than 7 T. However, as the strength of the static magnetic field increases, the wavelength of the radio-frequency field decreases and its amplitude distribution within the body to be imaged becomes less homogeneous.

Radio-frequency field inhomogeneity already introduces significant artifacts at 3 T. At 7 T, the Larmor frequency of protons is about 300 MHz, which corresponds to a wavelength around 14 cm in the human brain, i.e. a size comparable to that of a human head. In these conditions, the radio-frequency field B₁ is so inhomogeneous that images e.g. of a human brain obtained with standard techniques can become very difficult to interpret.

The radio-frequency (or “B₁”) inhomogeneity problem is so important that it could hinder further developments of high-resolution MRI. Moreover, the static magnetic field B₀ also shows a certain spatial inhomogeneity, which in turn induces artifacts. This effect is also worsened by the current trend of increasing the strength of the magnetic field. A number of techniques have been developed in order to deal with these inhomogeneity problems.
Composite pulses, i.e. cascades of elementary pulses parameterized by a phase and a flip angle (FA). The idea is to exploit symmetries in order to cancel errors at higher and higher orders while increasing the number of pulses and changing their FA and phases in a predetermined way. The problem is that they require in general large flip angles, therefore large energies and hence present potential problems for patient safety. See e.g. reference R1.
Adiabatic pulses: pulses whose amplitude and phase are continuously varied and slowly enough so that the spins evolve while staying aligned (or anti-aligned) with the effective magnetic field. This result follows from the adiabatic theorem in quantum mechanics. By changing slowly enough the amplitude and phase of the RF field, the spins follow with the same speed the direction of the effective field. A rotation of the spins can therefore be implemented in a robust way since it is mostly the rate of variation of the field that matters and not its value itself. These pulses were then developed further to be robust against B₀ inhomogeneity. The same problem as with the composite pulses occurs: they require long durations and large powers. Hence their use is limited in in-vivo applications and at high field. See e.g. reference R2.
Parallel transmission: the technique consists of irradiating the region of interest through the use of N, ideally independent, coils. Each one of them has its own inhomogeneity profile in reception and emission. If the amplitude and phases of these profiles are known, usually obtained via previous measurements, then an RF solution on each of the N coils can be designed to obtain either a homogeneous RF field over a region of interest or a homogeneous excitation pattern. The first option was baptised RF-shimming: see e.g. reference R3. The second option is known under the name “transmit SENSE”: see e.g. reference R4. The two techniques have a great potential. Two important drawbacks are that the high cost of the necessary equipment and the difficulty of dealing with RF safety aspects.
Strongly modulating pulses: they are trains of elementary pulses, or “subpulses”, each having a constant frequency and amplitude, and a continuous linear phase. These pulses were originally developed to provide good coherent control for a system of multiple coupled spins for Nuclear Magnetic Resonance Quantum Information Processing. See: reference R5 and reference R6. Strongly modulating pulses have also been used in MRI in order to counteract radio-frequency field inhomogeneity, particularly in high-field applications: see reference R7, as well as International Application WO 2009/053770.

One important drawback, in MRI, of the strongly modulating pulses is the fact that they are not spatially selective. Except for some relatively minor deviations of the resonance frequency due to different susceptibilities in the tissue or some imperfect B₀ shimming, the Larmor frequency does not vary in space since no magnetic field gradients are applied. Even if such gradients were applied, still strongly modulating pulses would not be suitable for spatially selective MRI because their spectra show strong sidelobes, due to the square shape of the elementary pulses. At the same time, the use of square elementary pulses allows finding an analytical solution of the Schrödinger equation for the nuclear spins, thus avoiding lengthy numerical calculation which would make application of strongly modulating pulses impractical.

The lack of spatial selectivity means that 3D reading techniques are necessary to obtain an artefact-free image and avoid aliasing or folding effects which would make the final image useless. In contrast, spatially selective techniques are advantageous because they allow a considerably faster acquisition of data, so that high resolution images can be obtained in a very reasonable time for a patient.

The invention aims at providing a spin excitation technique allowing compensation of B₁ and/or B₀ inhomogeneity and providing with spatial (“slice”) selectivity while retaining the advantageous features of the strongly modulating pulses.

The inventive technique uses a train of sub-pulses which are not square as in prior art strongly modulating pulses, but are instead suitable for performing slice-selective excitation when associated with magnetic gradients. Like in the prior art methods, the amplitudes, frequencies and initial phases of the sub-pulses are chosen in order to compensate for field inhomogeneity within the volume of interest. Because the shape of the RF sub-pulses is not square anymore, there is no general analytical expression to calculate the evolution of the spin system; therefore, it would seem that a lengthy numerical solution of the Schrödinger equation is necessary. But this is not necessarily true: the present inventors have discovered that, under appropriate conditions, such a “modified” strongly modulating pulse is approximately equivalent to a “conventional” strongly modulating pulse consisting of square sub-pulses. This allows a dramatic simplification of the pulse design process: one can simply design a conventional strongly modulating pulse using known methods, and then find the equivalent modified pulse allowing slice-selective excitation. Advantageously, an iterative refinement of the analytical solution can also be performed.

The invention described here does not require use of parallel transmission, and therefore allows avoiding the associated increase of cost. However, it could be combined with parallel transmission to achieve even better performances.

An object of the invention is then a method of performing nuclear magnetic resonance imaging of a body, comprising:
immerging said body in a static magnetic field for aligning nuclear spins along a magnetization axis;
exposing said body to a gradient pulse and to a transverse radio-frequency pulse for performing slice-selective excitation of said nuclear spins, thus flipping the nuclear spins of atoms contained within a slice of said body;
detecting a signal emitted by excited nuclear spins; and
reconstructing a magnetic resonance image of said slice of the body on the basis of the detected signal;

the method being characterized in that it comprises the steps of:

(i) designing a reference radio-frequency pulse suitable for performing, in the absence of a gradient pulse, non-slice selective excitation of said nuclear spins, said reference radio-frequency pulse being a “conventional” strongly modulating pulse, i.e. a composite pulse consisting of a train of elementary square pulses with constant frequencies; the number of elementary pulses, their frequencies and their initial phases being chosen in order to compensate for spatial inhomogeneity of said radio-frequency pulse at least within said slice of the body;

(ii) designing a transverse radio-frequency pulse by replacing each elementary square pulse of said reference radio-frequency pulse by a respective slice-selective elementary pulse having a same frequency and a same average amplitude; and

(iii) applying said transverse radio-frequency pulse to said body, together with a composite gradient pulse consisting of a train of respective elementary gradient pulses having an average amplitude equal to zero.

Advantageously, step (ii) can further comprise a sub-step of adjusting the amplitudes, frequencies and initial phases of said slice-selective elementary pulses in order to improve the homogeneity of the nuclear spin excitation through said slice of the body.

Preferably both said slice-selective elementary pulses and said elementary gradient pulses exhibit temporal symmetry.

According to particular embodiments of the invention:
All said elementary gradient pulses may have a same amplitude, except for sign.
Said elementary gradient pulses may have alternating polarities.
All said slice-selective elementary pulses and elementary gradient pulses may have a same duration.

Said step (i) of designing a reference radio-frequency pulse may be performed according to the algorithm described in above-referenced document WO 2009/053770, applied to the selected slice of the body to be imaged. In short, this algorithm comprises

(i-a) determining a statistical distribution of the amplitude of said radio-frequency pulsed field within said slice of the body;

and

(i-b) computing a set of optimal parameters of said reference radio-frequency pulse for jointly minimizing a statistical dispersion of the spin flip angles distribution within said slice of the body, and the errors between the actual spin flip angles and a predetermined target value thereof, said parameters comprising: the number of said elementary pulses, as well as the amplitude, frequency and initial phase of each of them.

Optionally, the algorithm can also comprise a sub-step (i-a′) of determining a statistical distribution of the amplitude of said static magnetic field along said magnetization axis within said slice of the body. In this case, step (i-b) of computing a set of optimal parameters of said reference radio-frequency pulsed field should be performed by taking into account said statistical distribution of the amplitude of said static magnetic field.

In any case, said sub-step (i-b) of computing a set of optimal parameters of said reference radio-frequency pulsed field is preferably performed by taking into account a penalty function depending on at least one of: the duration of the reference radio-frequency pulse, its peak power, its energy, its maximum frequency and its specific absorption rate.

The method of designing the reference pulse is not an essential part of the invention, and any alternative method could be used. For example, design could be based on the spatial distribution of the flip angle instead of its statistical distribution, although this would require a much greater computational effort. As it will be explained later, this spatial approach is indeed necessary when parallel transmission is used.

Indeed, in a particular embodiment of the invention, a plurality of transmit channels are used for exposing said body to a transverse radio-frequency pulse, each of said channels being characterized by a different radio-frequency field spatial distribution, and wherein said reference radio-frequency pulse and said transverse radio-frequency pulse consist of a superposition of components associated to respective transmit channels.

In this case, said step (i) can comprise:

(i-α) determining a spatial distribution of the amplitude and phase of the radio-frequency field transmitted by each of said transmit channels within said slice of the body; and

(i-β) computing a set of optimal parameters of said reference radio-frequency pulse for jointly minimizing a statistical dispersion of the spin flip angles distribution within said slice of the body, and the errors between the actual spin flip angles and a predetermined target value thereof,

said parameters comprising: the number of said elementary pulses, as well as the amplitude, frequency and initial phase of each of them and for each of said transmit channels.

Another object of the invention is a magnetic resonance imaging scanner comprising:
a magnet for generating a static magnetic field for aligning nuclear spins of a body to be imaged along a magnetization axis;
means for generating transverse radio-frequency pulses and gradient pulses, and for directing said pulses toward said body in order to perform slice-selective excitation of said nuclear spins; and
means for detecting a signal emitted by flipped nuclear spins within said slice of the body, and for reconstructing an image of said slice;

characterized in that said means for generating radio frequency and gradient pulses, and said means for detecting a signal and reconstructing an image are adapted for carrying out a method as described above.

Additional features and advantages of the present invention will become apparent from the subsequent description, taken in conjunction with the accompanying drawings, which show:

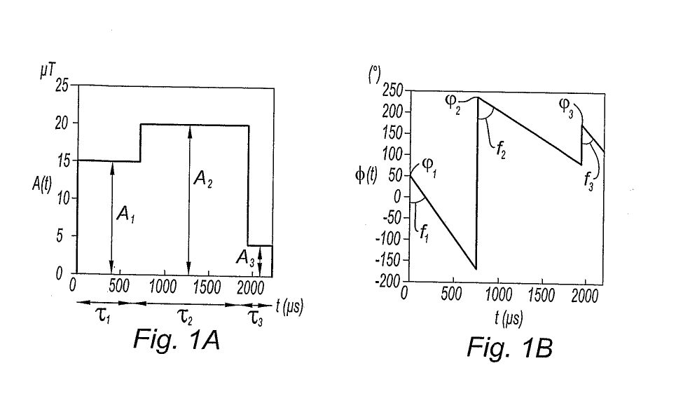

FIGS. 1A and 1B, the time-varying amplitude and phase of a conventional strongly modulating pulse;

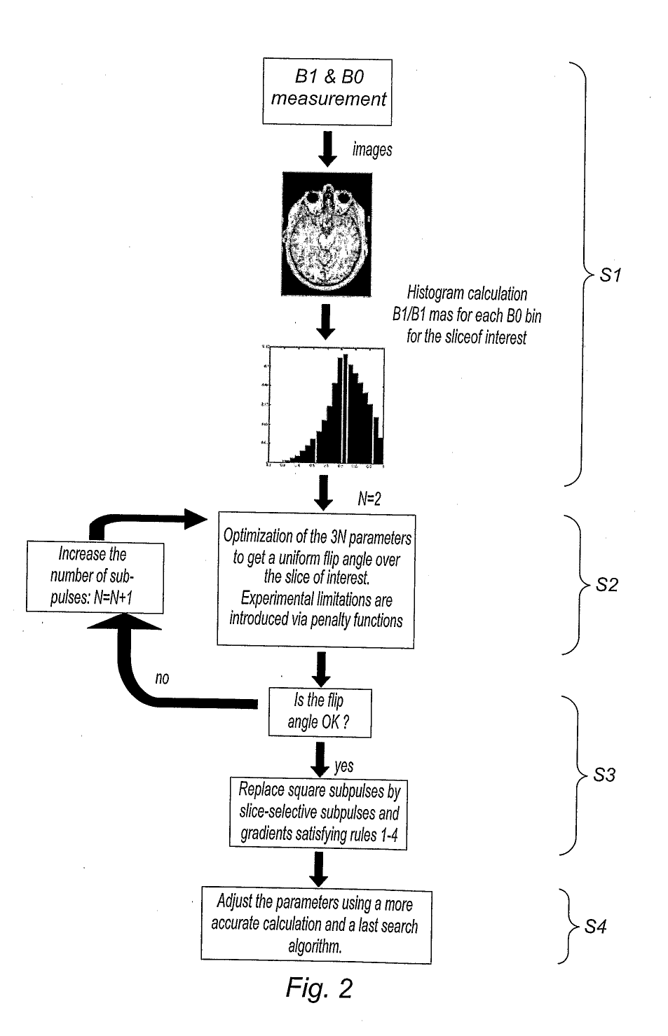

FIG. 2, a flow-chart of a pulse design method according to the invention;

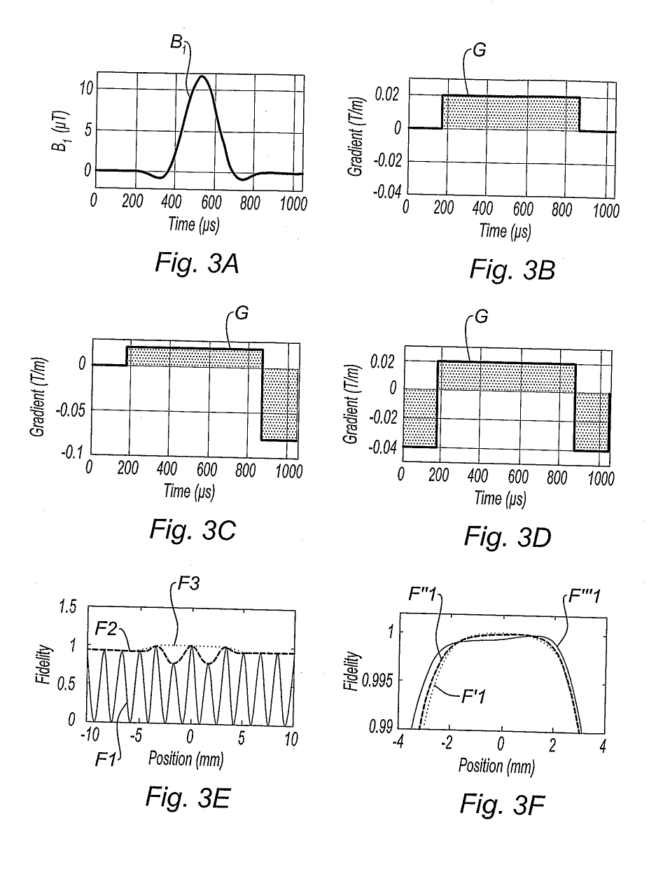

FIGS. 3A-3F, the results of numerical simulation illustrating the principle of the invention;

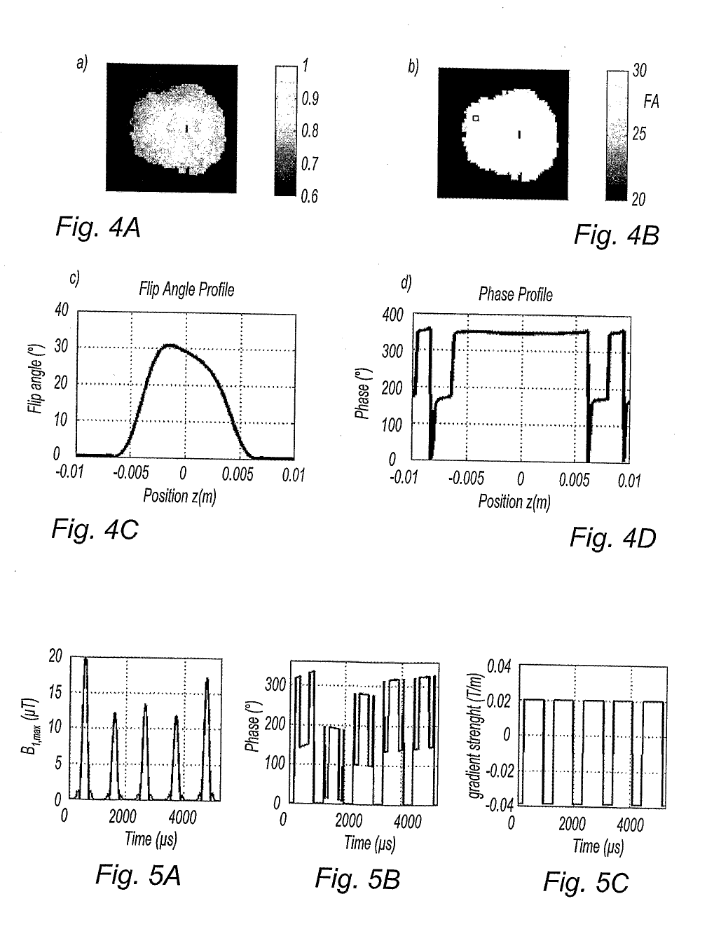

FIGS. 4A-4D numerical data illustrating the technical result of the invention;

FIGS. 5A-5C, gradient and RF pulses used for obtaining the data of FIGS. 4A-4D;

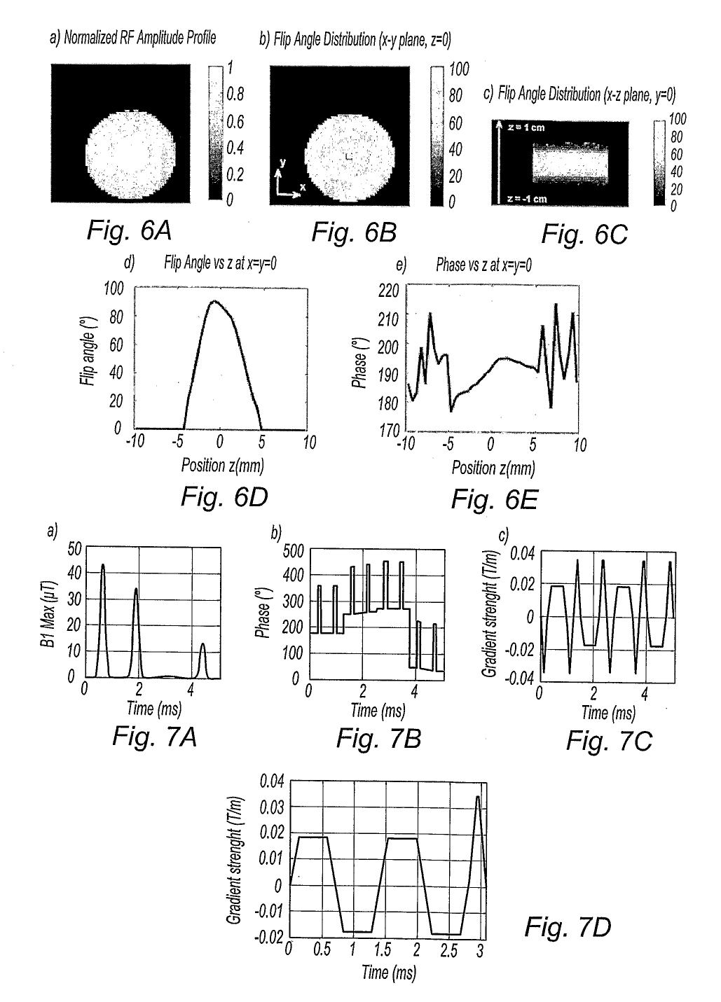

FIGS. 6A-6E, experimental data, also illustrating the technical result of the invention;

FIGS. 7A-7C, gradient and RF pulses used for obtaining the data of FIGS. 6A-6E;

FIG. 7D, an alternative but equivalent succession of gradient pulses; and

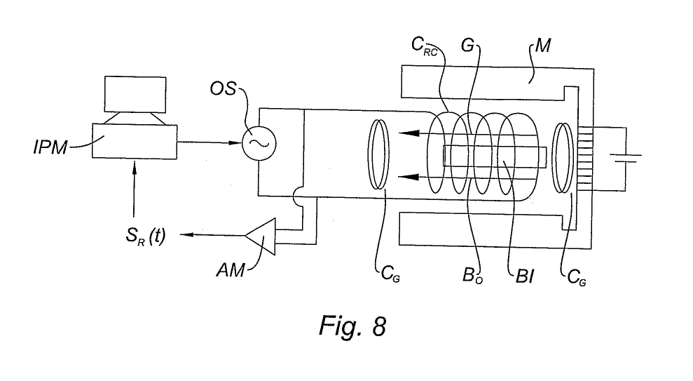

FIG. 8 a magnetic resonance imaging scanner according to an embodiment of the invention.

As known in the prior art, e.g. from above-referenced document WO 2009/053770, a strongly modulating pulse consists of a train of N elementary radio-frequency pulses of duration τᵢ, having a constant angular frequency ωᵢ and amplitude Aᵢ, and a continuous phase Φᵢ(t)=ωᵢ·t+φᵢ, with i=1−N. FIGS. 1A and 1B represent the time-dependent amplitude and phase of such a pulse composed by N=3 elementary pulses, or “sub-pulses”. It can be understood that a strongly modulating pulse is completely defined by a set of 4N parameters (τᵢ, Aᵢ, ωᵢ, φᵢ) with i=1−N. The values of these parameters are chosen in order to obtain a relatively uniform spin flip angle despite the unavoidable B₀ and B₁ inhomogeneity. Design of strongly modulating pulses is eased by the fact that, within the duration τᵢ of each elementary pulse, the phase of the radio-frequency field varies linearly with time, Φᵢ(t)=ωᵢ·t+φᵢ, Therefore, an analytical solution of the Schrödinger equation for the spins exists, which allows performing calculations in a reasonable time.

Document WO 2009/053770 describes an algorithm for designing strongly modulating pulses. A modified form of this algorithm can be applied to design slice-selective pulses according to the present invention. This modified algorithm is illustrated by the flow-chart of FIG. 2.

The algorithm begins with a preliminary calibration step, which consists in determining the maximum value, with respect to position \vec{r}, of the radio-frequency pulsed field amplitude B₁(\vec{r}) within the volume of the body to be imaged, or at least within the slice of interest. This allows normalization of the RF pulse amplitudes in the subsequent steps.

Then (step S1), a statistical distribution of the normalized amplitude of the radio-frequency pulsed field within the slice of interest of the body to be imaged is determined. This is a first difference with the algorithm described in WO 2009/053770, where the whole volume of interest (and not only a slice thereof) is considered. The slice can have any orientation in space.

The B₁ profile measurement can be performed using the method described in reference R8.

The statistical distribution can take the form of a one-dimensional or of a bi-dimensional histogram depending on whether only the B₁ or both the B₁ and B₀ inhomogeneity are taken into account.

The second step (S2) consists in determining the optimal shape of a strongly modulating pulse for jointly optimizing
the dispersion of the spin flip angles distribution within the slice of interest, e.g. the standard deviation σ_{FA} of the FA-distribution; and
the errors between the actual spin flip angles FA and their predetermined target value FA₀, e.g. the mean error of the FA: [symbol] |FA−FA₀| [symbol] .

Indeed, it is not only necessary to homogenize the FA distribution, which is quantified by σ_{FA}, but to homogenize it at the right value, which is expressed by [symbol] |FA−FA₀| [symbol] .

Moreover, the optimization has to be carried out under a number of constraints, which depend on both the hardware and the body to be imaged (e.g. a human patient, which cannot be exposed to an arbitrarily high RF power): overall duration of the composite pulse (Στᵢ), its peak power, its energy, its maximum frequency, its specific absorption rate, etc. These constraints can be expressed by a penalty function contributing to the “cost function” to be minimized by the optimization procedure, F( [symbol] |FA−FA₀| [symbol] ,σ_{FA}).

A second difference with the algorithm described in WO 2009/053770 is that, in the case of the present invention, the sub-pulses are taken of a same duration τ. This is related to the need of performing slice-selective excitation: it is known that spatial selectivity is related to the spectral width of the RF pulse which, in turn, is related to its duration. If the RF elementary pulses had different durations, it would be necessary to modify the corresponding gradient pulses in order to compensate for their different spectral width and ensure a uniform selectivity. This would unduly complicate the design algorithm.

The optimization step (S2) can be carried out iteratively, as follows:
First of all, a minimum number N of elementary pulses is predetermined; usually N=5;
Then optimal values for the amplitudes Aᵢ, frequencies ωᵢ and relative initial phases φᵢ of said elementary pulses are determined, and the corresponding values of [symbol] |FA−FA₀| [symbol] and σ_{FA} are computed. Optimization consists in minimizing a cost function such as F( [symbol] |FA−FA₀| [symbol] ,σ_{FA})=α [symbol] |FA−FA₀| [symbol] +βσ_{FA}/ [symbol] FA [symbol] +PF, with e.g. α=0.4 and β=1.6, PF representing the above-mentioned penalty function expressing the constraints on the composite pulses.
The errors between the actual spin flip angles and their predetermined target value, [symbol] FA−FA₀| [symbol] and the dispersion of the spin-flip angles distribution σ_{FA} are then compared to respective threshold values, ε, δ, and/or the cost function F is compared to a single threshold T. If these comparisons show that the optimal strongly modulating pulse for the present value of N is satisfactory, the optimization step ends. Otherwise the value of N is increased by 1 and optimization is repeated.

The strongly modulating pulse obtained at the end of step S2 is not spatially selective, and is not used directly. Rather, it serves as a “reference” pulse for designing a slice-selective selective pulse. This is performed in step S3, wherein each square sub-pulse is replaced by an “equivalent” slice-selective sub-pulse.

As it is known in the field of MRI, a slice-selective RF pulse has a spectrum which is approximately square (of course, a pulse with a perfectly square spectrum is not physically feasible); it can be e.g. a “sinc” (cardinal sinus) pulse apodized by a smooth window such as a Hanning window. Such a pulse is not slice-selective “per se”. It only allows slice selective excitation when it is applied to a body to be imaged together with a magnetic field gradient G perpendicular to the slice to be selected. The magnetic field gradient is also pulsed; therefore, the expression “gradient pulse” will be used in the rest of this document.

A slice-selective RF pulse coupled with a gradient pulse is considered “equivalent” to a square pulse when it induces—within the slice of interest—approximately the same evolution of the nuclear spins. It is not obvious that an equivalent slice-selective pulse can be found for an arbitrary square pulse (with constant frequency i.e. linearly-varying phase). It is even less obvious that such an equivalent pulse can be found without having to solve numerically the Schrödinger equation for the nuclear spins. A quantum mechanical demonstration of this unexpected fact will be provided later. For the time being, only the rules for finding the equivalent slice-selective RF pulse of each square sub-pulse of the “reference” strongly modulating pulse will be provided. These rules are the following:

Rule 1: both elementary pulses must have a same (constant) frequency, and a same initial phase (relative to other elementary pulses of the corresponding composite pulse).

Rule 2: the time average of the envelopes of both pulses has to be the same:

$$\frac{1}{T} {\int }_{0}^{T} {B}_{1}^{ref} ( t ) d t = \frac{1}{T} {\int }_{0}^{T} {B}_{1}^{s . s .} ( t ) d t$$

where T is the duration of the pulses, B₁^{ref}(t)=constant is the magnetic field of the reference square sub-pulse and B₁^{s.s.}(t) is the magnetic field of the equivalent slice-selective elementary pulse.

Rule 3: the time average of the gradient pulse has to be zero:

$$\frac{1}{T} {\int }_{0}^{T} G ( t ) d t = 0$$

Rule 4: both the RF elementary pulse B₁^{s.s.}(t) and the gradient pulse G(t) exhibit temporal symmetry.

In practice, B₁^{s.s.}(t) will be appreciably different from zero only in the central part of the time interval T (see FIG. 3A); G(t) will be chosen constant in said central part, with sidelobes of opposite polarity to make its temporal average equal to zero (see FIG. 3D).

Rules 1 to 3 are essential, while rule 4 is not.

This illustrated by FIGS. 3A-3D, showing the result of numerical simulations.

FIG. 3A shows the envelope (in μT, or microTesla) of a slice-selective RF pulse whose shape is defined by a “sinc” function apodized by a Hanning window. The carrier frequency of the pulse is constant, and equal to the Larmor frequency of the nuclei to be excited; its bandwidth is 6 kHz. The spatially-selective excitation performed by said pulses (when associated with an appropriate gradient pulse) will be compared to that obtained by a square pulse having the same initial phase (φ₀=0), the same carrier frequency and a same average amplitude (rules 1 and 2 are satisfied). More precisely, the amplitude of the square reference pulse—and the average amplitude of the selective pulse—are chosen in order to induce a spin flip angle of π/6:

FA=πc/6=γ·T·B₁^{ref}(t)=γ∫₀^{T}B₁^{s.s.}(t)dt

where γ is the gyromagnetic ratio of the nuclei. Indeed, it is well known that for a square RF pulse at the resonance (Larmor) frequency, having constant amplitude B₁ and duration T, the flip angle is given by γ·T·B₁.

FIGS. 3B to 3D show three gradient pulses which can be associated with the RF pulse of FIG. 3A. In all cases, the amplitude of the gradient pulse (or, at least, of its central part) is 20 mT/m. This amplitude and the spectral bandwidth of the RF pulse determine the thickness of the slice of the body in which nuclear spins are excited. Here, the slice thickness (defined as the full width at half maximum of the spin flip angle) is taken equal to 7 mm. In this specific example, the magnetic field gradient is oriented along the z axis, i.e. the magnetization axis.

The gradient pulse of FIG. 3B has nonzero average; therefore it does not comply with rules 3. The gradient pulse of FIG. 3C does have zero average, but it is not symmetric with respect to temporal inversion; therefore it complies with rule 3 but not with rule 4. The gradient pulse of FIG. 3D complies with both rule 3 (zero average) and rule 4 (symmetry).

FIG. 3E shows the “gate fidelity” between the propagator U describing the action of the slice-selective RF pulse plus a gradient pulse, and the propagator U^{th} of the corresponding square pulse. The “propagator” is the operator describing the temporal evolution of a quantum system. “Gate fidelity” is a metric that was introduced in quantum information processing to quantify how close two unitary operations can be (see reference R5) It is given by: Fid=|trace(U^{th}U^{†})/2|², where U^{†} is the hermitian conjugate of U.

Curve F1 corresponds to the first scenario, where the gradient pulse of FIG. 3B is used. It can be seen that the gate fidelity oscillates strongly, its average value is of the order of 0.5 and it is near to 1 only for specific point in the z-coordinate (z=0 corresponding to the center of the slice). Therefore, when rules 3 and 4 are violated, the “slice-selective” pulse has an effect which is very different from the “reference” square pulse.

Curve F2 corresponds to the second scenario, where the gradient pulse of FIG. 3C is used. Oscillations are weaker, and the average fidelity is higher. It can be said that the “slice selective” pulse is approximately equivalent to the “reference” one.

Curve F3 corresponds to the third scenario, where the gradient pulse of FIG. 3C is used. Fidelity remains above 0.995 for −2 mm≦z≦2 mm. The equivalence between the “slice-selective” and the “reference” pulses is quite satisfactory.

This equivalence is preserved even if the B₀ magnetization field is not perfectly homogeneous. FIG. 3F shows the gate fidelity in the third scenario assuming a homogeneous B₀ field (curve F′1), a field inhomogeneity ΔB₀=100 Hz (curve F″1) and of ΔB₀=200 Hz (curve F′″1). As customary in the field of MRI, magnetic fields are expressed in units of frequency (the conversion factor between B₀ and the Larmor frequency being γ). It can be seen that fidelity remains high for −2 mm≦z≦2 mm even for ΔB₀=200 Hz.

It can be understood that, if every sub-pulse of the reference strongly modulating pulse found at the end of step S2 is replaced by a slice-selective RF pulse satisfying rules 1, 2 (and preferably 4) associated with a gradient pulse satisfying rule 3 (and preferably 4), slice-selective excitation is obtained while preserving the inhomogeneity-compensation effect characterizing strongly modulating pulses. The composite RF pulse obtained at the end of step S3 of the algorithm of FIG. 2 can be directly applied to MRI. In a preferred embodiment, however, this composite pulse will be used to initialize a final search algorithm to adjust its parameters by computing the true quantum mechanical evolution of the spins at z=0 (step S4). This optional adjustment or refinement step (S4 on the flow-chart of FIG. 2) can be performed using a line-search algorithm (see reference R9) or another direct technique such as gradient descent. This refinement step is performed quickly, because the composite pulse used for initializing it is already a good guess.

The technical result of the invention has been demonstrated by taking a measured B₁ profile in a human brain at 3 T and designing a 30° pulse using the algorithm previously discussed. FIG. 4A shows the measured normalized B₁ profile at 3 T with which the {B₁, B₀} histogram was calculated. With the parameters returned, a waveform was created. FIG. 4B shows the result of the full numerical simulation of the flip angle at z=0 (position where the spins do not see a magnetic field gradient). FIGS. 4C and 4D show the simulated flip angle and phase along the slice thickness (z direction) for the voxel indicated by a square in 4B. The phase is pretty flat over the slice while the flip angle is quite uniform compared to the uncompensated profile.

The pulse (amplitude and phase) and gradient waveforms to achieve such a result are given in FIGS. 5A, 5B and 5C. No refinement step has been implemented.

An experimental validation of the method of the invention was performed using a Siemens Magnetom 7 T scanner, with a volume coil, and an 8-cm diameter sphere filled with distilled water and 5 g of NaCl, serving as a phantom. The B₁ profile measurement was performed using the method described in R8. This method actually allows measuring the flip angle for a given voxel. However, for a pulse at resonance, the flip angle is merely the integral of the pulse with respect to the time so that the B₁ field value can be easily calculated from that measurement. The B₀ measurement was done using the same sequence, but by inserting a second gradient echo in the first TR (TR1) to determine the phase evolution of the spins between the two echoes. It was set TR=400 ms (n=5) and a resolution of 2×2×3 mm³ and a matrix size of 64×64×40. FIG. 6A shows the measured normalized RF field amplitude over the central axial slice. There is roughly a factor of 2 between the centre and the periphery of the slice. With the values of B₁ and B₀ on that slice only, a bi-dimensional histogram was calculated and fed into the optimization algorithms to design a slice selective strongly modulating pulse with target flip angle of 90°. The program returned for z=0, i.e. for the exact centre of the slice which sees no magnetic field gradient, a simulated mean flip angle of 90.3° and a standard deviation of 4.27°, which yields a ratio std/mean=4.7%, compared to the original 13.5% given by the RF inhomogeneity profile. The pulse lasted 5.06 ms and is given in FIG. 7A (amplitude) and FIG. 7B (phase). The gradient pulse is provided in FIG. 7C. The target gradient strength value during the RF pulse was 18 mT/m. Each sub-RF pulse was a sinc-function apodized with a Hanning window, with duration 700 μs and bandwidth of 4 kHz.

It is interesting to note that, by switching sequentially the polarity of the gradients (as in FIG. 7C), the compensating lobes cancel each other and can therefore be removed (see FIG. 7D, where only the final lobe is present), thereby reducing the duration of the overall pulse.

To confirm the performance of the pulses, the returned RF and gradient pulses were inserted in the sequence for measuring the flip angles. Two versions of this measurement were implemented: one with the gradient during the pulse, and one without. When the gradient is turned on, spins within the slice thickness respond slightly differently. As shown in reference R10, the flip angle measurement can incorporate a bias since what is really measured is an integrated effect over the slice, while the calculation is done for a single z position. The second version, without the gradient pulse, allowed to get rid of this bias, removing the possibility of an imperfect implementation of the gradient shapes (due to eddy currents for instance). For the first version, the results are shown in FIG. 6B through FIG. 6E. To reduce a possible bias discussed above, a 3D reading was still performed with partition thickness of 0.5 mm. Based on an estimate of the T1, an error of 1 to 3 degrees in the flip angle is expected. For the first measurement, and the slice of interest, the mean flip angle was measured to be 82.2°, and a standard deviation of 6.4°, thus yielding std/mean=7.8%. In the second version, without the gradient pulse during the RF, the mean was found to be 88.1°, std=4.9°, and thus std/mean=5.6%, values closer to theoretical predictions.

A particularly advantageous feature of the method of the invention is that it can be carried out by a conventional scanner provided with suitable information processing means. Such a conventional scanner is schematically represented on FIG. 8. It comprises: a magnet M for generating a static magnetic field B₀ in which is immersed a body BI to be imaged; a coil C_{RF} for irradiating said body by a transverse radio-frequency pulse B₁ and for detecting signal emitted by flipped nuclear spins within said body; coils C_{G} for generating magnetic field gradients along three perpendicular axis x, y and z (on the figure, for the sake of simplicity, only coils for generating a gradient along the z-axis have been shown), electronic means (an oscillator) OS for generating the radio-frequency pulse, an amplifier AM for amplifying said spin resonance signal before digitizing it, and information processing means IPM. The information processing means IPM receive and process the amplified resonance signal S_{R}(t) and, most importantly, controls the oscillator OS, determining the shape, energy, phase and frequency of the RF-pulse. A scanner according to the present invention is characterized in that said information processing means IPM are adapted for carrying out a method as described above. Since the information processing means IPM are usually based on a programmable computer, software means (executable code stored in a computer memory device) can turn a standard scanner into a device according to the invention, without any need for hardware modifications.

On FIG. 8, a single RF coil is used for both transmission and reception; however, these functions can also be performed by separate coils. Moreover, several transmit RF coils can be used to allow parallel transmission.

It is possible to provide a proof of the fact that application of rules 1-4 leads to slice-selective pulses which are approximately equivalent to square “reference” pulses. The proof is based on average Hamiltonian theory, described in reference R11.

A spin is located at a z position, in a magnetization field B₀(r) directed along the z-axis. The magnetization field comprises a uniform component B̅₀ and a (unwanted) spatially-varying component, ΔB₀(r).

A RF pulse with a time-varying amplitude B₁(t), an initial phase φ₀ and a frequency S2 is applied, together with a magnetic field gradient G along the z direction. The RF carrier frequency can be written: Ω=ω_{L}+ω, where ω_{L} is the Larmor frequency of the spin in the uniform magnetization field B̅₀.

The Hamiltonian for the spin, in a frame rotating at the Larmor frequency is:

$$H ( \vec{r} , t ) = - \frac{γ ( Δ {B}_{0} ( r ) + G ( t ) z )}{2} {σ}_{z} - \frac{γ {B}_{1} ( r , t )}{2} ( {σ}_{x} cos ( {φ}_{0} + ω t ) + {σ}_{y} sin ( {φ}_{0} + ω t ) \qquad [ 1 ]$$

where γ is the gyromagnetic ratio (in rad/T) and σᵢ are the Pauli matrices.

In the frame rotating at the carrier frequency Ω=ω_{L}+ω, the Hamiltonian becomes:

$${H}_{rot} ( \vec{r} , t ) = - \frac{γ ( Δ {B}_{0} ( r ) + G ( t ) z ) + ω}{2} {σ}_{z} - \frac{γ {B}_{1} ( r , t )}{2} ( {σ}_{x} cos ( {φ}_{0} ) + {σ}_{y} sin ( {φ}_{0} ) ) \qquad [ 2 ]$$

which is still time-dependent via G(t) and B₁(t). Because the Hamiltonian does not commute with itself at all times, there is no analytical solution, even at z=0, unless ΔB₀=ω=0. However the evolution can be formally expressed by the following propagator:

$$U ( \vec{r} , t ) = {e}^{- i ω {σ}_{z} T / 2} {T}_{Dyson} {e}^{i {\int }_{0}^{T} {H}_{rot} ( t ) d t} \qquad [ 4 ]$$

where T_{Dyson} is the Dyson time-ordering operator. If B₁ and G were time-independent, T_{Dyson} would simply be the identity matrix and one would recover the previous solution, i.e. the one for the non-selective strongly modulating pulses. Equation [3] can be recast as:

$$U ( \vec{r} , t ) = {e}^{- iω {σ}_{z} T / 2} {T}_{Dyston} {e}^{i {\int }_{0}^{T} [ ( ω + γ Δ {B}_{0} + γ G ( t ) z ) {σ}_{z} + γ {B}_{1} ( r , t ) ( {σ}_{x} co s ( {φ}_{0} ) + {σ}_{y} si n ( {φ}_{0} ) ) ] / 2 d t} \qquad [ 3 ]$$

which, in turn can be rewritten as

U(\vec{r},T)=e^{-iωσ}^{_{z}}^{T/2}e^{iH}^{_{AV}}^{T} [5]

Where

$${H}_{AV} = {H}^{( 0 )} + {H}^{( 1 )} + {H}^{( 2 )} + … \\ {H}^{( 0 )} = \frac{1}{T} {\int }_{0}^{T} {H}_{rot} ( t ) d t \\ {H}^{( 1 )} = - \frac{i}{2 T} {\int }_{0}^{T} d {t}_{2} {\int }_{0}^{{t}_{2}} d {t}_{1} [ {H}_{rot} ( {t}_{2} ) , {H}_{rot} ( {t}_{1} ) ] \\ {H}^{( 2 )} = \frac{1}{6 T} {\int }_{0}^{T} d {t}_{3} {\int }_{0}^{{t}_{3}} d {t}_{2} {\int }_{0}^{{t}_{2}} d {t}_{1} ( \begin{matrix}[ \begin{matrix}{H}_{rot} ( {t}_{3} ) , \\ [ \begin{matrix}{H}_{rot} ( {t}_{2} ) , \\ {H}_{rot} ( {t}_{1} )\end{matrix} ]\end{matrix} ] + \\ [ {H}_{rot} ( {t}_{1} ) , {H}_{rot} ( {t}_{3} ) ]\end{matrix} )$$

This is called the Magnus expansion (see reference R11) and Hₐᵥ is the average Hamiltonian. Proof of this series and its convergence can for instance be found in reference R12.

It is important to note that all of these terms are time-independent. H⁽⁰⁾ is called the zero order term of the average Hamiltonian, H⁽¹⁾ is the first order term and so on. Here the term H⁽⁰⁾ is simply given by

$$H ( \vec{r} , t ) = - \frac{γ ( Δ {B}_{0} ( r ) + G ( t ) z )}{2} {σ}_{z} - \frac{γ \vert {B}_{1 , Tot} ( r , t ) \vert}{2} ( {σ}_{x} cos ( {φ}_{T} + ω t ) + {σ}_{y} sin ( {φ}_{T} + ω t ) ) \qquad [ 9 ]$$

It can be seen that if the integral of G(t) is zero, then, to zero order:

$${H}^{( 0 )} = \frac{1}{2} {σ}_{z} ( ω + γ Δ {B}_{0} + \frac{1}{T} γ z {\int }_{0}^{T} G ( t ) d t ) + \frac{1}{2} ( {σ}_{x} cos {ϕ}_{0} + {σ}_{y} sin {ϕ}_{0} ) \frac{1}{T} {\int }_{0}^{T} γ {B}_{1} ( r , t ) d t \qquad ( 6 )$$

which is independent of z (except via B₁, but this can be neglected over the slice thickness). To zero order, the action of the gradient is cancelled so that the propagator U(r,T) does not depend on z. Moreover, B₁(t) contributes only through its time average.

Equation [7] resembles closely to the well-known analytical propagator for a square pulse (constant B₁), which is expressed by:

U(\vec{r},t)=e^{-ωσ}^{_{z}}^{t/2}e^{i((ω+γΔB}^{₀}^{)σ}^{_{z}}^{+γB}^{₁}^{(σ}^{ₓ}^{cos(φ}^{₀}^{)+σ}^{_{y}}^{sin(φ}^{₀}^{)))t/2} [7′].

Indeed, equation [7] is identical to [7′], except in that B₁ is replaced by

$$U ( r , T ) \approx {e}^{- i ω {σ}_{z} T / 2} {e}^{i {H}^{( 0 )} T} = {e}^{- i ω {σ}_{z} T / 2} {e}^{i ( ( ω + γ Δ {B}_{0} ) {σ}_{z} + ( {σ}_{x} co s {ϕ}_{0} + {σ}_{y} si n {ϕ}_{0} ) \frac{1}{T} {\int }_{0}^{T} γ {B}_{1} ( r , t ) d t ) T / 2} \qquad [ 7 ]$$

It can then be asserted that the time-varying RF pulse B₁(t), associated with a suitable gradient pulse G, is equivalent to zero order to a square pulse in the vicinity of z=0 provided that both RF pulses have a same carrier frequency, initial phase and average amplitude (rules 1 and 2), and that the average gradient is zero (rule 3).

However, zero-order approximation is often unsatisfactory, as the term H⁽¹⁾ in the Magnus expansion can be significant.

It can be shown that if H(t)=H(T−t), i.e. if the Hamiltonian has time reflection symmetry, then H⁽¹⁾=H⁽³⁾= . . . =H⁽ⁿ⁾=0, for any odd n. If the RF elementary pulse, B₁(t), and the associated gradient pulse, G(t), have time reflection symmetry, so has the Hamiltonian. Therefore equation [7] is valid up to second order. This justifies the optional rule 4. Furthermore, it can be shown that H⁽²⁾ introduces a correction which modifies only the scalar terms in the exponential of Eq. [7].

To the appropriate level of approximation (zero order or second order, depending on the fact that rule 4 is applied or not), spins in the slice behave in a same way. Moving away from the centre of the slice, the G term becomes larger, and so does the H⁽²⁾ term, and the approximation starts breaking down. The spins remote from the centre get barely affected by the RF, thus making the pulse slice selective. The excitation profile is then expected to be the inverse Fourier transform of the RF pulse, as explained by reference R13.

As mentioned above, the use of “modified” strongly modulating pulses can optionally be combined with parallel transmission. Indeed, if parallel transmission is available, one extension of the pulse design technique described above consists in determining, for each emitting channel, the initial phases φ_{k,n} and amplitudes B_{1,k,n} of each elementary RF pulse. Here k refers to the channel index while n refers to the elementary pulse index. In this procedure, the B₁ field distribution varies from one elementary pulse to another since it directly depends on the interference pattern corresponding to the phases and amplitudes set on the different channels. Calculating the performance of a pulse candidate hence can not be done with the help of a statistical distribution of the spin flip angles (e.g. a one- or two-dimensional histogram), but requires computing the flip angle for every voxel. For each elementary pulse, the number of degrees of freedom therefore is greatly increased: M amplitudes, M initial phases (M being the number of channels), and one frequency. For a single elementary pulse, the number of parameters now is 2M+1 (=17 for a commonly encountered 8 channels system). If N is the number of elementary pulses, the number of degrees of freedom now is N(2M+1), e.g. 85 for 8 channels and 5 elementary pulses.

The mathematical description of the spin evolutions is the same as before except that now the B₁ field is the result of the superposition and interference between the different contributions arising from the different channel elements. Let a spin be located at a z position. A pulse with amplitude B_{1,k}(t), an initial phase φ_{0,k} and a frequency ω is applied on channel #k; a magnetic field gradient G along the z direction is also applied.

The total field B_{1,Tot} at a position r now is given by:

$$\frac{1}{T} {\int }_{0}^{T} γ {B}_{1} ( r , t ) d t .$$

The Hamiltonian for a spin sitting at this location is:

$${B}_{1 , Tot} ( r , t ) = {\sum }_{k = 1}^{M} {B}_{1 , k} ( r , t ) {e}^{i {ϕ}_{O , k}} = \vert {B}_{1 , Tot} ( r , t ) \vert {e}^{i {ϕ}_{T}} \qquad [ 8 ]$$

Now the Hamiltonian looks exactly the same as equation [1] above. Indeed, equation [1] corresponds to a special case of equation [9], where on each channel is sent an identical pulse shape, up to a phase and a scaling factor, resulting in a time-independent phase φ_{T} of the total field.

Individual complex B₁ maps corresponding to the different channels are required to allow computing the total RF field. In the optimization, for each sub-pulse, the algorithm aims at determining the optimal complex scaling factors of the basic waveforms (e.g. apodized sinc shapes) on each channel. These scaling factors return a B_{1,Tot}. But as these factors may vary from one elementary pulse to the next, the evolution needs to be computed on every voxel (or at least a large fraction of them). In other words, the statistical approach described in WO 2009/053770 and which leads to a very significant simplification of the optimization problem in the single-channel case has to be replaced by a more burdensome spatial approach.

### REFERENCES

R1: M. H. Levitt and R. R. Ernst, “Composite pulses constructed by a recursive expansion procedure”, Journal of Magnetic Resonance, vol. 55, pp 247 (1983).
R2: R. S. Staewen, A. J. Johnson, B. D. Ross, T. Parrish, H. Merkle and M. Garwood, “3D flash imaging using a single surface coil and a new adiabatic pulse, BIR-4”, Invest. Radiol., vol. 25, 559-567 (1990).
R3: P. F. Van de Moortele, C. Akgun, G. Adriany, S. Moeller, J. Ritter, CM Collins, M. B. Smith, J. T. Vaughan, K. Ugurbil, “B1 destructive interferences and spatial phase patterns at 7 T with a head transceiver array coil”, Magnetic resonance in medicine, vol. 54, pp 1503-1518 (2005).
R4: U. Katscher, P. Bornert, C. Leussler, J S van den Brink, “Transmit SENSE”, Magnetic Resonance in Medicine, vol. 49, pp 144-150 (2003).
R5: E. M. Fortunato, M. A. Pravia, N. Boulant, G. Teklemariam, T. F. Havel and D. G. Cory, “Design of strongly modulating pulses to implement precise effective Hamiltonians for quantum information processing”, Journal of Chemical Physics, vol. 116, pp 7599-7606 (2002)
R6: M. A. Pravia, N. Boulant, J. Emerson, A. Farid, E. Fortunato, T. F. Havel, R. Martinez, D. G. Cory, “Robust control of quantum information”, Journal Chemical Physics 119, pp 9993-10001 (2003)
R7: N. Boulant, D. Le Bihan and A. Amadon “Strongly modulating pulses to counteract RF inhomogeneity at high fields”, Magnetic Resonance in Medicine, 60:701-705, 2008
R8: V. L. Yarnykh, “Actual Flip-Angle Imaging in the Pulsed Steady State: A Method for Rapid Three dimensional Mapping of the Transmitted Radiofrequency field”, Magnetic Resonance in Medicine, vol. 57, pp 192-200 (2007).
R9: M. J. D. Powell, “Direct search algorithms for optimization calculations” Acta Numerica (1998), 7:287-336 Cambridge University Press
R10: X. Wu, D. K. Deelchand, V. L. Yarnykh, K. Ugurbil, and P-F. Van de Moortele, “Actual Flip Angle Imaging: from 3D to 2D”, Proceedings of the 17^{th} ISMRM meeting, p 372, Honolulu, Hi., USA (2009).
R11: U. Haeberlen and J. S. Waugh, Coherent Averaging Effects in Magnetic Resonance; Physical Review 175, 2, p 453 (1968).
R12: P. Pechukas, and J. C. Light, “On the exponential form of the time-displacement operator in quantum mechanics”, Journal of Chemical Physics 44, p 3897 (1966).
R13: J. Pauly, D. Nishimura, and A. Macovski, “A linear class of large-tip-angle selective excitation pulses”, Journal of Magnetic Resonance 82, p 571 (1989).

1. A method of performing nuclear magnetic resonance imaging of a body (BI), comprising:
immerging said body in a static magnetic field (B₀) for aligning nuclear spins along a magnetization axis;
exposing said body to a gradient pulse (G) and to a transverse radio-frequency pulse (B₁) for performing slice-selective excitation of said nuclear spins, thus flipping the nuclear spins of atoms contained within a slice of said body;
detecting a signal emitted by excited nuclear spins; and
reconstructing a magnetic resonance image of said slice of the body on the basis of the detected signal;
the method further comprising the steps of:
(i) designing a reference radio-frequency pulse suitable for performing, in the absence of a gradient pulse, non-slice selective excitation of said nuclear spins, said reference radio-frequency pulse being a composite pulse consisting of a train of elementary square pulses with constant frequencies; the number of elementary pulses, their frequencies and their initial phases being chosen in order to compensate for spatial inhomogeneity of said radio-frequency pulse at least within said slice of the body;
(ii) designing a transverse radio-frequency pulse by replacing each elementary square pulse of said reference radio-frequency pulse by a respective slice-selective elementary pulse having a same frequency and initial phase, and a same average amplitude;
(iii) applying said transverse radio-frequency pulse to said body, together with a composite gradient pulse consisting of a train of respective elementary gradient pulses having an average amplitude equal to zero.

2. A method according to claim 1 wherein step (ii) further comprises a sub-step of adjusting the amplitudes, frequencies and initial phases of said slice-selective elementary pulses in order to improve the homogeneity of the nuclear spin excitation through said slice of the body.

3. A method according to claim 1 wherein said slice-selective elementary pulses and said elementary gradient pulses exhibit temporal symmetry.

4. A method according to claim 1 wherein all said elementary gradient pulses have a same amplitude, except for sign.

5. A method according to claim 1 wherein said elementary gradient pulses have alternating polarities.

6. A method according to claim 1 wherein all said slice-selective elementary pulses and elementary gradient pulses have a same duration.

7. A method according to claim 1 wherein said step (i) comprises:
(i-a) determining a statistical distribution of the amplitude of said radio-frequency pulse within said slice of the body; and
(i-b) computing a set of optimal parameters of said reference radio-frequency pulse for jointly minimizing a statistical dispersion of the spin flip angles distribution within said slice of the body, and the errors between the actual spin flip angles and a predetermined target value thereof, said parameters comprising: the number of said elementary pulses, as well as the amplitude, frequency and initial phase of each of them.

8. A method according to claim 7, further comprising a sub-step (i-a′) of determining a statistical distribution of the amplitude of said static magnetic field along said magnetization axis within said slice of the body, and wherein said sub-step (i-b) of computing a set of optimal parameters of said reference radio-frequency pulsed field is performed by taking into account said statistical distribution of the amplitude of said static magnetic field.

9. A method according to claim 7, wherein said sub-step (i-b) of computing a set of optimal parameters of said reference radio-frequency pulsed field is performed by taking into account a penalty function depending on at least one of: the duration of the reference radio-frequency pulse, its peak power, its energy, its maximum frequency and its specific absorption rate.

10. A method according to claim 1, wherein a plurality of transmit channels are used for exposing said body to a transverse radio-frequency pulse (B₁), each of said channels being characterized by a different radio-frequency field spatial distribution, and wherein said reference radio-frequency pulse and said transverse radio-frequency pulse (B₁) consist of a superposition of components associated to respective transmit channels.

11. A method according to claim 10, wherein said step (i) comprises:
(i-α) determining a spatial distribution of the amplitude and phase of the radio-frequency field transmitted by each of said transmit channels within said slice of the body; and
(i-β) computing a set of optimal parameters of said reference radio-frequency pulse for jointly minimizing a statistical dispersion of the spin flip angles distribution within said slice of the body, and the errors between the actual spin flip angles and a predetermined target value thereof, said parameters comprising: the number of said elementary pulses, as well as the amplitude, frequency and initial phase of each of them and for each of said transmit channels.

12. A magnetic resonance imaging scanner comprising:
a magnet for generating a static magnetic field for aligning nuclear spins of a body to be imaged along a magnetization axis;
means for generating transverse radio-frequency pulses and gradient pulses, and for directing said pulses toward said body in order to perform slice-selective excitation of said nuclear spins; and
means for detecting a signal emitted by flipped nuclear spins within said slice of the body, and for reconstructing an image of said slice;
wherein said means for generating radio frequency and gradient pulses, and said means for detecting a signal and reconstructing an image are adapted for carrying out a method according to claim 1.
